(19) United States
(12) Patent Application Publication
(10) Pub. No.: US 2010/0178703 A1
(43) Pub. Date: Jul. 15, 2010

(54) SINGLE-SENSOR METER SYSTEM WITH NO SENSOR HANDLING AND METHOD OF USING THE SAME

(75) Inventors: John P. Creaven, Granger, IN (US); Andrew J. Dosmann, Granger, IN (US); Jason R. Diehl, Kendallville, IN (US); William W. McCloud, Elkhart, IN (US)

Correspondence Address: NIXON PEABODY LLP, 300 S. Riverside Plaza, 16th Floor, CHICAGO, IL, 60606-6613, US

(73) Assignee: Bayer Healthcare LLC, Tarrytown, NY (US)

(21) Appl. No.: 12/530,803

(22) PCT Filed: Mar. 12, 2007
(86) PCT No.: PCT/US07/06172

§ 371 Date: Mar. 4, 2010

(51) Int. Cl. G01N 35/00; G01N 33/48; G01N 27/00; G01N 21/00; G01N 21/75

(52) U.S. Cl. 436/43; 422/68.1; 422/82.01; 422/82.05; 436/164

(57) ABSTRACT

A single-sensor meter system for dispensing sensors for testing of an analyte concentration in a fluid comprises a container assembly and a single-sensor meter. The container assembly includes inner and outer cartridges. The inner cartridge includes a plurality of test sensors and a mechanical mechanism. The container assembly forms exactly one opening for dispensing the test sensors one at a time. The opening is covered by an end cap so as to assist in preventing or inhibiting moisture from entering the interior of the container assembly. The mechanical mechanism is adapted to advance the plurality of test sensors. The single-sensor meter is adapted to align with and operatively connects to the container assembly. The single-sensor meter includes a test-sensor extraction mechanism adapted to grip a test sensor and pull the test sensor through the opening to a dispensed position.

### FIELD OF THE INVENTION

The invention generally relates to a single-sensor meter system and method of using the same. More specifically, the invention is directed to a single-sensor meter system that does not involve a user handling a test sensor and a method of the same.

### BACKGROUND OF THE INVENTION

The quantitative determination of analytes in body fluids is of great importance in the diagnoses and maintenance of certain physiological abnormalities. For example, lactate, cholesterol and bilirubin should be monitored in certain individuals. Additionally, determining glucose in body fluids is important to diabetic individuals who must frequently check the glucose level in their body fluids to regulate the glucose intake in their diets. The results of such tests can be used to determine how much, if any, insulin or other medication needs to be administered. In one type of blood-glucose testing system, test sensors are used to test a sample of blood.

Existing testing is performed in conjunction with test sensors (test strips) and a meter or instrument. Handling of the test sensors can be a challenge for many users of the meter or instrument. One way of reducing or eliminating the handling of test sensors is to have meters that are multi-sensor meters that include a cartridge. Meters with cartridges, however, tend to be larger, heavier, more mechanically complicated and somewhat less portable than single-sensor meters. Existing single-sensor meters, however, need an alternative packaging of test sensors in, for example, a bottle or single-foil test sensors. Bottles can be both bulky and present challenges for users in removing a single test sensor at a time. Single-foil test sensors, while extremely portable, can be a significant challenge to open for users with reduced manual dexterity.

Accordingly, it would be desirable to have a single-sensor meter system that addresses these problems.

### SUMMARY OF THE INVENTION

According to one embodiment, a single-sensor meter system for dispensing sensors for testing of an analyte concentration in a fluid comprises a container assembly and a single-sensor meter. The container assembly includes an inner cartridge and an outer cartridge. The inner cartridge includes a plurality of test sensors and a mechanical mechanism. The container assembly forms exactly one opening for dispensing the test sensors one at a time. The opening is covered by an end cap so as to assist in preventing or inhibiting moisture from entering the interior of the container assembly. The mechanical mechanism is adapted to advance the plurality of test sensors. The single-sensor meter is adapted to align with and operatively connects to the container assembly. The single-sensor meter includes a test-sensor extraction mechanism adapted to grip a test sensor and pull the test sensor through the opening to a dispensed position.

According to one method, a single-sensor meter system is operated to determine or analyte concentration of a fluid. The method comprises providing a container assembly including an inner cartridge and an outer cartridge. The inner cartridge includes a plurality of test sensors and a mechanical mechanism. The container assembly forms exactly one opening for dispensing the test sensors one at a time. The opening is covered by an end cap so as to assist in preventing or inhibiting moisture from entering the interior of the container assembly. The mechanical mechanism is adapted to advance the plurality of test sensors. A single-sensor meter including a test-sensor extraction mechanism is provided. The end cap is moved from the closed position to the open position. The container assembly and the single-sensor meter are aligned. One of the plurality of test sensors from the container assembly is retrieved such that one of the test sensors is at least partially located within the single-sensor meter. The analyte concentration is determined.

### BRIEF DESCRIPTION OF THE DRAWINGS

FIG. 1a is a front view of a container assembly according to one embodiment of the invention.

FIG. 1b is a side perspective view of the container assembly of FIG. 1a without the end cap and without the sidewall.

FIG. 1c is a side view of the container assembly of FIG. 1a with the end cap.

FIG. 1d is a side view of a container assembly according to another embodiment with an end cap in a closed position.

FIG. 1e is a side view of the container assembly of FIG. 1d with the end cap in an open position.

FIG. 1f is a side view of a container assembly according to a further embodiment with a slidable end cap in a closed position.

FIG. 1g is a side view of the container assembly of FIG. 1f with the slidable end cap in an open position.

FIG. 2a is a front perspective view of a single-sensor meter according to one embodiment of the invention with the slider in a first position.

FIG. 2b is a front view of the single-sensor meter of FIG. 2a with the slider being moved from the first position.

FIG. 2c is a front view of the single-sensor meter of FIG. 2a with the slider in a second position.

FIG. 2d is a top view of the single-sensor meter of FIG. 2b.

FIG. 3a is a top partial perspective view of a single-sensor meter system using the container assembly of FIGS. 1a-1c and the single-sensor meter of FIGS. 2a-2d.

FIG. 3b is a side partial perspective view of the single-sensor meter system of FIG. 3a.

FIG. 4 is a front perspective view of the single-sensor meter of FIG. 2a with a test sensor in the first position and ready for testing.

FIG. 5 is a front perspective view of a single-sensor meter according to another embodiment of the invention.

### DETAILED DESCRIPTION OF THE ILLUSTRATED EMBODIMENTS

The present invention is directed to a single-sensor meter system that includes a container assembly and a single-sensor meter or instrument. The container assembly and the single-sensor meter or instrument are adapted to be aligned with and operatively connected to each other to form the single-sensor meter system. The container assembly includes a plurality of test sensors. The present invention is advantageous in that a user can transfer a test sensor from the container assembly to the single-sensor meter without having to handle the test sensor.

The plurality of test sensors is used to determine concentrations of analytes. Analytes that may be measured using the present invention include glucose, lipid profiles (e.g., cholesterol, triglycerides, LDL and HDL), microalbumin, hemoglobin A_{1c}, fructose, lactate, or bilirubin. The present invention is not limited, however, to these specific analytes and it is contemplated that other analyte concentrations may be determined. The analytes may be in, for example, a whole blood sample, a blood serum sample, a blood plasma sample, or other body fluids like ISF (interstitial fluid) and urine.

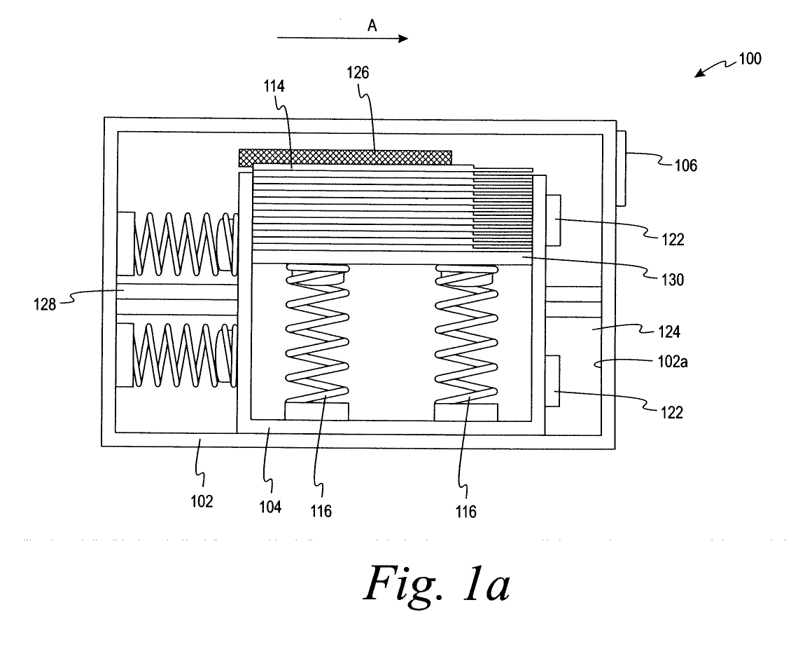

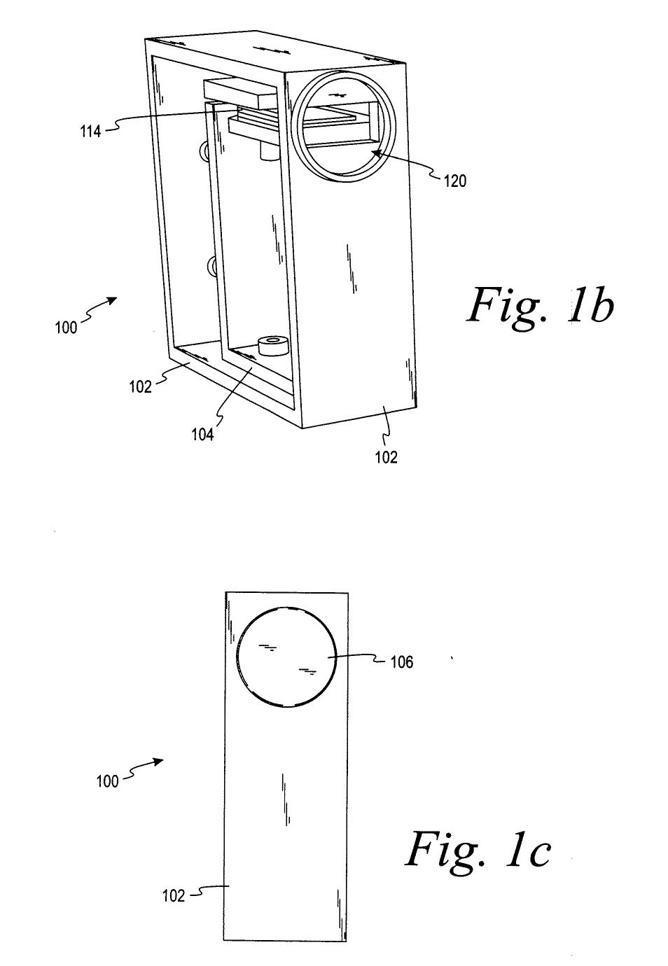

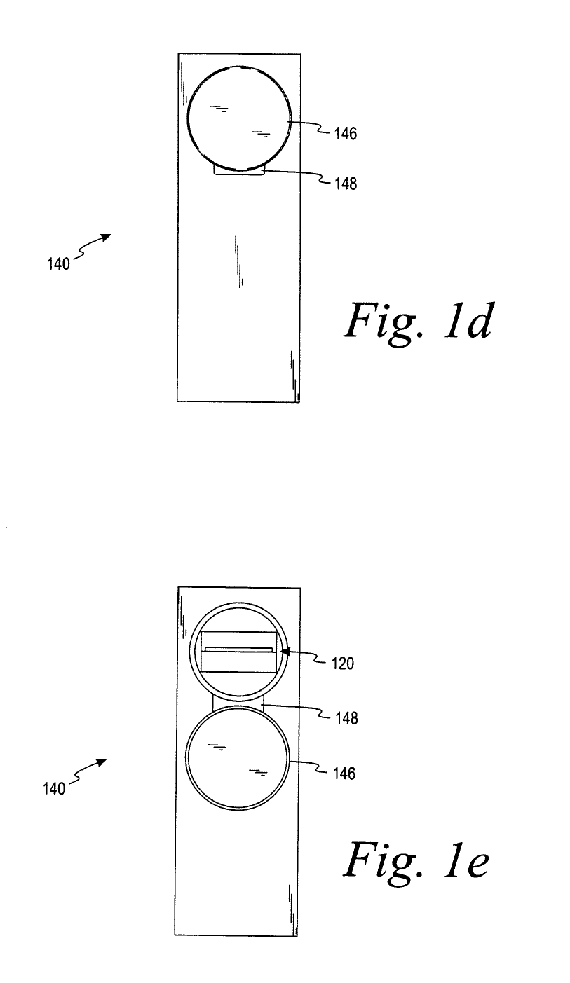

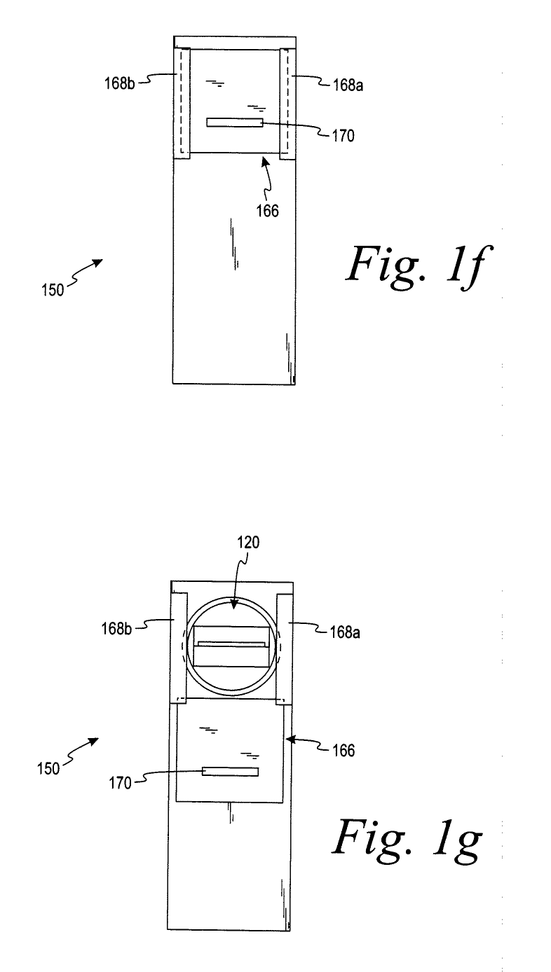

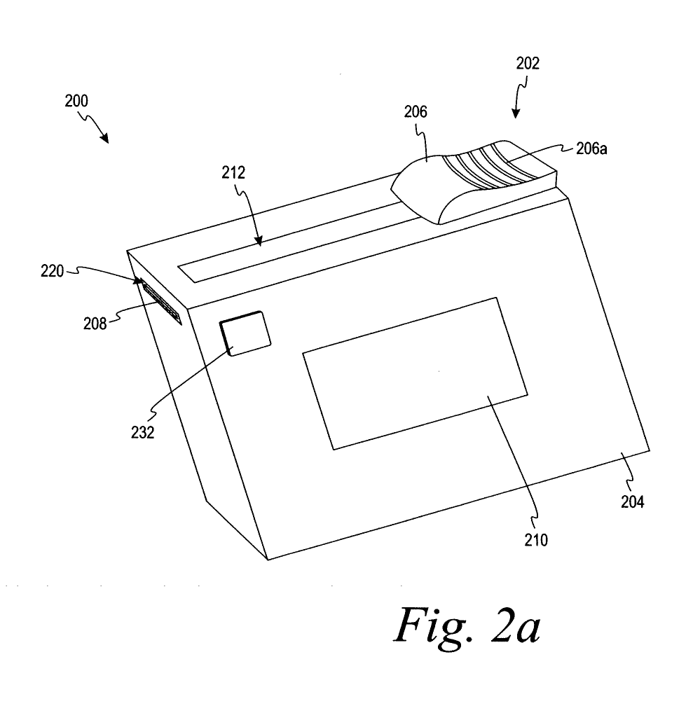

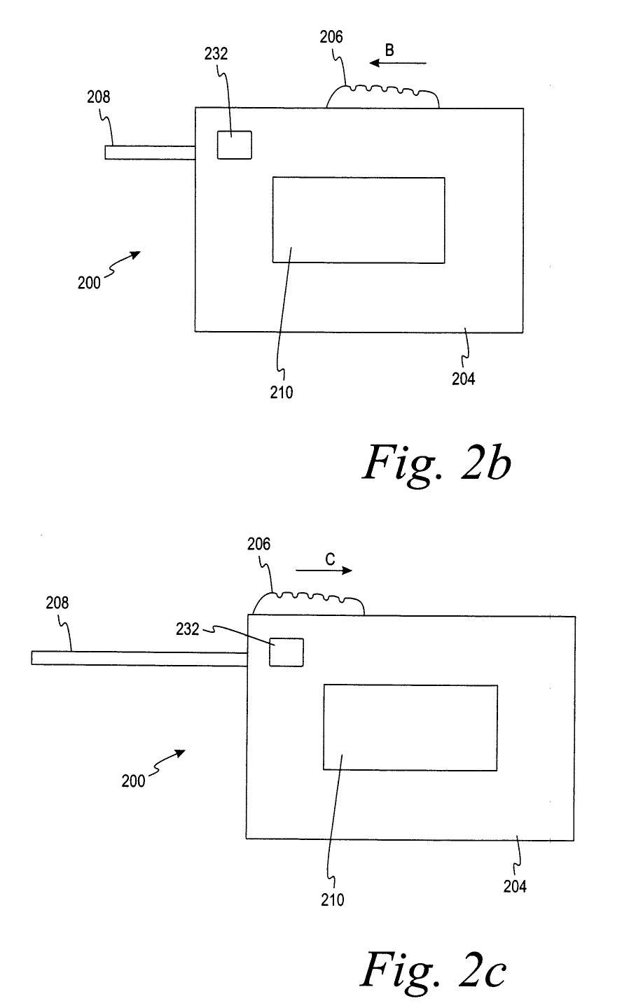

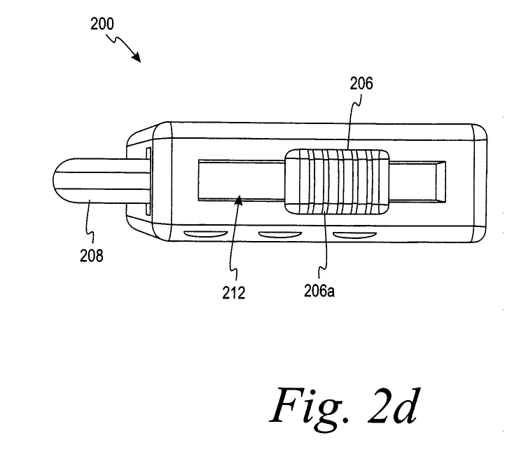

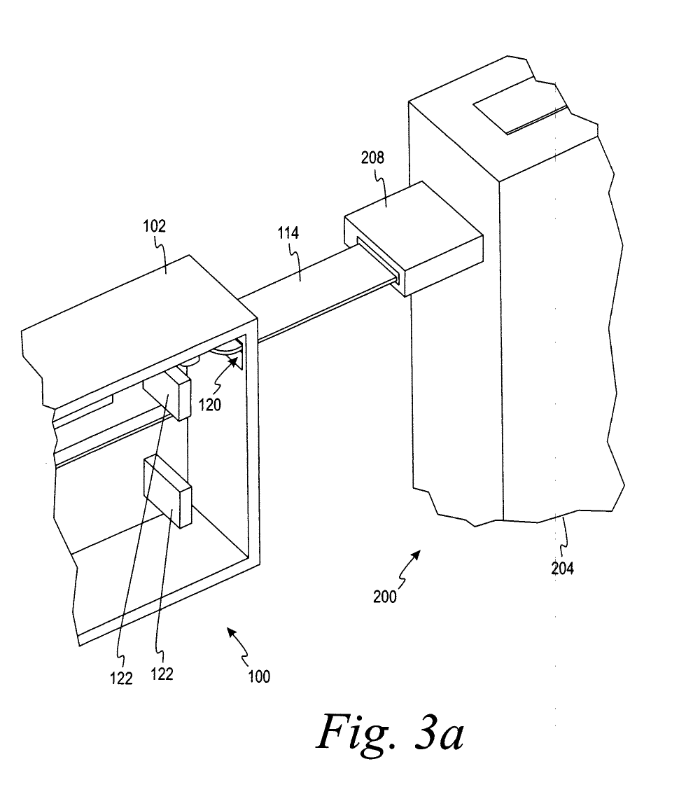

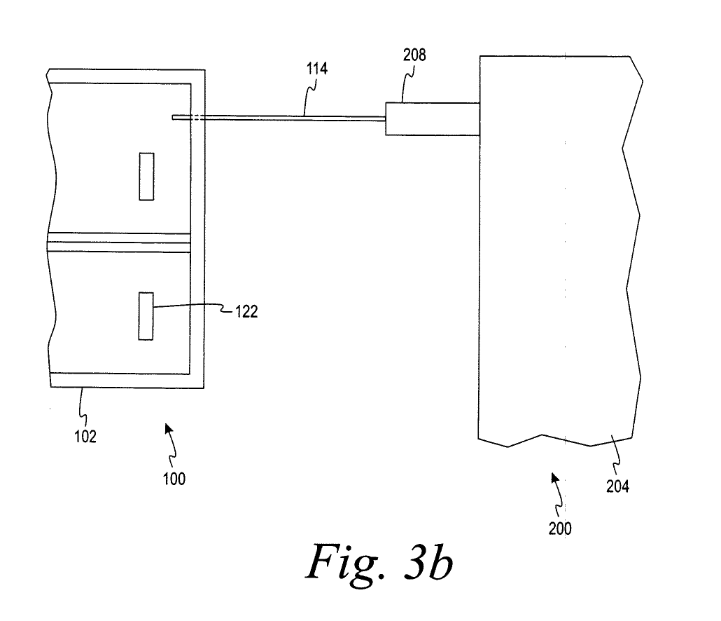

Referring to FIGS. 1a-c, a container assembly 100 is shown that is adapted to be used with a single-sensor meter or instrument 200 (FIGS. 2a-2d). The container assembly 100 is adapted to be aligned with and operatively connected to the single-sensor meter or instrument 200 as shown in FIGS. 3a,3b.

The container assembly 100 is desirably a substantially moisture-proof and air-tight device. The container assembly 100 of FIGS. 1a-c comprises an outer cartridge 102, an inner cartridge 104 and an end cap 106. The inner cartridge 104 comprises a plurality of test sensors 114 and a mechanical mechanism 116 (FIG. 1a). The container assembly 100 is adapted to be disposable after each of the plurality of test sensors 114 has been used in one embodiment. It is contemplated, however, in another embodiment, that an inner cartridge 104 with unused test sensors may replace the inner cartridge without test sensors. In this embodiment, the inner cartridge is disposable while the outer cartridge is adapted to be re-used.

Referring to FIG. 1b, the outer cartridge 102 forms an opening 120 through which the test sensors 114 exit the container assembly 100. To better show the opening 120, FIG. 1b shows the container assembly 100 without the end cap 106 of FIG. 1a. In the closed position (see, e.g., FIGS. 1a, 1c), the opening 120 is covered and desirably sealed with the end cap 106. The end cap 106 assists in preventing or inhibiting air and moisture from entering into the interior of the inner cartridge 104 that contains the plurality of test sensors 114. The opening 120 extends from the outer cartridge 102 to the inner cartridge 104. The opening 120 is sized to allow the plurality of test sensors 114 to move therethrough one at a time and eventually exit the inner cartridge 104 and outer cartridge 102.

The inner surface of the outer cartridge 102 as shown in FIG. 1a includes at least one stopping member 122. The at least one stopping member 122 prevents the movement of the inner cartridge 104 when the at least one stopping member 122 contacts an outer surface of the inner cartridge 104. This prevents further movement of the inner cartridge 104 in the direction of the opening 120 (direction of arrow A in FIG. 1a). In addition, the space created between the inner cartridge 104 and outer cartridge 102 when the at least one stopping member 122 contacts the inner cartridge 104 may contain desiccant material 124. In another embodiment, further movement of the inner cartridge is prevented by an inner surface 102a of the outer cartridge 102. Thus, in this embodiment, the at least one stopping member is not present.

The outer cartridge 102 comprises a holding plate 126 that holds the upper most of the plurality of sensors 114 (as viewed in FIG. 1a) from moving, when the inner cartridge 104 is in motion. As will be further discussed below, during the operation of the single-sensor meter system, the inner cartridge 104 moves away from the direction of the opening 120. During this movement, the holding plate 126 contacts the upper most test sensor and holds the test sensor in place while the remainder of the stack of sensors 114 moves with the inner cartridge 104.

The movement of the inner cartridge 104 within the outer cartridge 102 is assisted by a guiding mechanism 128. The guiding mechanism 128 ensures that the inner cartridge 104 moves in a generally linear fashion during the operation of the single-sensor meter system. The guiding mechanism 128 has a generally triangular profile that provides a generally linear motion for the inner cartridge 104. It is contemplated that other guiding mechanisms may be used than that depicted in FIG. 1a.

As shown in FIG. 1a, the plurality of test sensors 114 is stacked within the inner cartridge 104. The plurality of test sensors 114 is adapted to assist in testing at least one analyte. As discussed above, one of the analytes that may be tested is glucose from, for example, a whole blood sample. In one embodiment, the plurality of test sensors 114 would include an appropriately selected enzyme to react with the desired analyte or analytes to be tested. An enzyme that may be used to react with glucose is glucose oxidase. It is contemplated that other enzymes may be used such as glucose dehydrogenase. An example of a test sensor 114 is disclosed in U.S. Pat. No. 6,531,040 assigned to Bayer Corporation. It is contemplated that other test sensors may be used in the container assembly 100.

The test sensors 114 may be electrochemically-based test sensors in one embodiment. In another embodiment, the test sensors 114 may be optical-based test sensors. In this embodiment, the instrument or meter would be adapted to read the optical-based test sensor in determining the concentration of the analyte.

In one embodiment, one of the test sensors 114 may be a calibration test sensor. The calibration test sensor is a test sensor that is adapted to calibrate for the reagent lot. Specifically, electrical contacts of the single-sensor meter in this embodiment read the code of the calibration test sensor and calibrate the meter for the test sensors. This type of calibration is an automated calibration. The calibration test sensor should be located as the uppermost test sensor (as viewed in FIG. 1a) such that the calibration is performed on all of the test sensors.

The plurality of test sensors 114 may vary in number than shown in FIG. 1a so as to address the needs of different users. Typically, the stacked test sensors contain from about 10 to about 100 sensors and, more specifically, contain from about 25 to about 50 sensors. Because of limited shelf- and use-life of the test sensors, it is envisioned that a user who tests infrequently would likely desire an inner cartridge having less test sensors compared to a user who tests more frequently.

To urge the stacked test sensors 114 upwardly, the mechanical mechanism 116 is used according to one embodiment. The mechanical mechanism 116 is located within the inner cartridge 104, and assists in positioning one of the plurality of test sensors for eventual ejection from the container assembly 100 via the opening 120. The mechanical mechanism is any device that can exert pressure on the test sensors 114 so as to position one of the plurality of test sensors for ejection.

For example, the mechanical mechanism 116 depicted in FIG. 1a comprises two springs and a sensor-pressure plate 130 that guides the stack of sensors in an upwardly manner. Various types of springs may be used as the mechanical mechanism to upwardly urge the test sensors 114. For example, the spring may be a compression spring or a torsion spring. Springs are desirable because of their simplicity and ease of use. In another embodiment, the mechanical mechanism may include at least one spring or a plurality of springs.

To assist in protecting the reagent(s) in the test sensors 114, desirable packaging material and/or desiccant material may be used. The container assembly 100 is typically packaged in material that prevents or inhibits air from entering into an interior of the inner cartridge 104 that contains the test sensors 114. One type of removable packaging that may be used to enclose the container assembly 100 is aluminum foil. It is contemplated that other types of removable packaging may be employed. It is contemplated that desiccant material may be added in the interior of the removable packaging to assist in maintaining an appropriate humidity level therein. If the reagent in the test sensors is not humidity sensitive, then there is little or no need to include much, if any, desiccant. The removable packaging with or without the desiccant material assists in increasing the shelf-use of the test sensors. The removable packaging is to be removed before the container assembly 100 is aligned with the single-sensor meter 200.

It is contemplated that the container assembly 100 may be initially placed in a polymeric container (not shown) such as a bottle or other type of container. The container may be shaped similarly to the container assembly with a desirable seal to prevent or inhibit air or moisture from entering the interior of the container. The container may include a lid that is attached to the remainder of the container via a living hinge. It is contemplated that desiccant may also be added within the container. The container with or without the desiccant material assists in increasing the shelf-use of the test sensors. The container assembly 100 is removed from the container before being aligned with and operatively connected with a single-sensor meter 200.

Desiccant material 124 is desirably added to the container assembly 100 to assist in maintaining an appropriate humidity level within the interior of the inner cartridge 104 that contains the test sensors 114. In certain embodiments, the desiccant material 124 may be added to the space between the outer cartridge 102 and inner cartridge 104 as exemplified in FIG. 1a. In another embodiment, the desiccant material may be added to other areas within the outer cartridge or the inner cartridge. Specifically, some moisture may enter the interior of the outer cartridge 102 when the end cap 106 is removed, but such moisture is desirably absorbed by the desiccant material 124 so as to protect the reagent in the test sensors from degradation. By maintaining an appropriate humidity level, reagent material in the test sensors is protected. The amount of desiccant material 124 should be sufficient to obtain the desired shelf-life (the time period before any of the plurality of test sensors are used). More specifically, the shelf-life typically refers to the time period before the container assembly 100 is removed from the packaging material, if used. The amount of desiccant material 124 should also be sufficient to obtain the desired use-life (the time period after first use of one of the plurality of test sensors). More specifically, the use-life typically refers to the time period after the container assembly 100 is removed from the packaging material, if used.

Examples of desiccant that may be included within the container assembly, the removable packaging enclosing the container assembly, or the container containing the container assembly 100 include commercially available desiccants. The desiccant may be in the form of several shapes including balls, tablets, granular, or paper. For example, the desiccant may be molecular sieve spheres or thick desiccant paper. A non-limiting example of desiccant material may be purchased from Multisorb of Buffalo, N.Y. in the form of, for example, molecular sieve beads. In certain embodiments of the invention, an inner surface of the outer cartridge may be coated with desiccant or alternately could be made of a desiccant material.

It is contemplated that desiccant may not be used for test sensors that are not humidity sensitive. The amount of desiccant used, if any, depends on how humidity sensitive the test sensor is and the duration of the desired use-life.

In a closed position, the end cap 106 desirably seals the interior of the container assembly 100 such that the environment and any moisture in it is prevented or inhibited from contacting the test sensors 114. In such a closed position, the end cap desirably provides a substantially moisture-proof and a substantially air-tight cartridge. The end cap 106 is desirably designed to prevent or inhibit moisture or other contaminants from entering via the opening 120 and affecting the plurality of test sensors 114 for at least the shelf-life and use-life of the plurality of sensors. When the end cap 106 is removed, the test sensors 114, one at a time, can be moved through the opening 120 so as to eventually exit via the opening 120.

It is also contemplated that an end cap may be pivoted away from the opening 120 such as shown in FIGS. 1d, 1e in another embodiment. Specifically, a container assembly 140 includes an end cap 146 of FIGS. 1d, 1e that pivots about a hinge 148 such that the test sensors 114, one at a time, can be moved through the opening 120 so as to eventually exit via the opening 120. It is contemplated that the end cap may be of different forms, shapes and sizes than depicted in FIGS. 1c-e.

For example, the end cap may be a slidable end cap that is adapted to move between open and closed positions. The end cap needs to be adapted to move between open and closed positions and cover the opening in the closed position. On such example is shown in FIGS. 1f, 1g with container assembly 150 in which an end cap 166 is a slidable end cap that moves between a closed position (FIG. 1f) and an open position (FIG. 1g). The end cap 166 assists in sliding between the open position and the closed positions by the détentes 168a,b. The end cap 166 may include a small extension 170 thereof to assist in moving the end cap between the open and the closed positions.

The outer and inner cartridges 102, 104 may be made of a variety of materials, but is typically made of polymeric material. Some examples of polymeric materials that may be used in forming the cartridges 102, 104 include polycarbonate, ABS, nylon, polystyrene, polypropylene, or combinations thereof. It is contemplated that other polymeric materials may be used in forming the cartridge 102, 104. Other additives may be added in forming the housing such as, for example, TEFLON® for lubrication or glass to provide strength. It is contemplated that other additives may be employed. Polycarbonate is desirable for several reasons including being a durable material and having an ability to prevent or inhibit air (especially oxygen and moisture) from entering the outer cartridge 102, which in turn can enter the inner cartridge 104. Additionally, if the outer cartridge is formed from two distinct sections, polycarbonate is capable of sealing to itself. This may be desirable in a process where the two cartridge sections are sonically welded.

The outer and inner cartridges 102, 104 may be formed by processes known to those skilled in the art including injection-molding processes. If injection-molding processes are used, the wall thicknesses are typically designed within normal ranges. It is contemplated that other processes may be used such as a molding process.

The end caps may be made of different materials such as, for example, polymeric materials. It is desirable for the end caps to be made of materials that has some flexibility to cover the formed opening.

Referring to FIGS. 2a-2d, the single-sensor meter or instrument 200 is depicted according to one embodiment. The single-sensor meter or instrument is used to determine concentrations of analytes. Analytes that may be measured using the present invention include glucose, lipid profiles (e.g., cholesterol, triglycerides, LDL and HDL), microalbumin, hemoglobin A_{1c}, fructose, lactate, or bilirubin. The present invention is not limited, however, to these specific analytes and it is contemplated that other analyte concentrations may be determined. The analytes may be in, for example, a whole blood sample, a blood serum sample, a blood plasma sample, or other body fluids like ISF (interstitial fluid) and urine.

The single-sensor meter 200 comprises a sliding assembly 202, and housing 204. As shown in FIG. 2a, the sliding assembly 202 includes a slider 206 and a test sensor-extraction mechanism 208 attached to the slider 206. As shown in FIGS. 3a,b, the housing 204 is adapted to align with the container assembly 100. The device housing may comprise an LCD screen 210 that displays analyte concentrations. The housing 204 of FIGS. 2a-2d aligns the container assembly 100 with the end of the housing 204 from which the test sensor-extraction mechanism 208 extends towards the container assembly 100. Instead of being a side-aligning device, the housing may be a bottom-aligning device in another embodiment.

It is contemplated that other cartridges and container assemblies may be used. Depending on the shape of the cartridge to be used, the interior of the device housing may be redesigned to better align with the shape of the container assembly.

Referring to FIG. 2a, the slider 206 is shown in a first position. By continuing to manually move the slider 206 in a forward direction (direction of arrow B in FIG. 2b), the slider 206 is moved to a second position as shown in FIG. 2c. The slider 206 in FIG. 2c is located closer to the container assembly 100 than the slider 206 of FIGS. 2a,2b.

The sliding assembly 202 is adapted to grip one of the plurality of test sensors 114 from the inner cartridge 104 and pull it at least partially through the opening 120, such as shown in FIGS. 3a, 3b. When the slider 206 is in the first position (FIG. 2a), the test sensor-extraction mechanism 208 (which is also in its first position in FIG. 2a) does not contact any of the plurality of test sensors 114 and is contained almost entirely with the housing 204. As the slider 206 is moved in a forward direction (see direction of arrow B in FIG. 2b), the test sensor-extraction mechanism 208 (see FIGS. 2b,2c) is also moved in a forward direction.

Referring back to FIG. 2a, the exterior: of the housing 204 forms an external channel 212 on the upper portion of the housing 204. To facilitate easy movement of the slider, the slider 206 of FIG. 2a is guided along the external channel 212 (see FIGS. 2a, 2d). The slider 206 is connected to the test sensor-extraction mechanism via a connecting mechanism (not shown), such that the movement of the slider 206 corresponds to the movement of the test sensor-extraction mechanism 208. To enable easier gripping by the user, the slider 206 may form ridges or serrations 206a on a top surface thereof such as shown in FIGS. 2a-c.

Referring back to FIG. 2a, the test sensor-extraction mechanism 208 is located in the internal channel 212 that assists in facilitating and guiding the movement and positioning of the test sensor-extraction mechanism 208 from a first position (FIG. 2a) to a second position (FIG. 2c) and back to the first position. The sliding assembly 202 also includes a guiding block (not shown) to further ensure that the test sensor-extraction mechanism 208 is moving in a proper plane. The guiding block is located below the slider 206, and moves along the internal channel 212 with the test sensor-extraction mechanism 208. In one embodiment, the guiding block is the connecting mechanism by which the slider 206 is connected to the test sensor-extraction mechanism 208.

According to one process, the test sensor-extraction mechanism 208 of FIG. 2a extends through an opening 220 in the housing and then extends towards and moves through the opening 120 and subsequently contacts the inner cartridge 104. After contacting the inner cartridge, the test sensor-extraction mechanism 208 continues to move forward until contacting one of the plurality of test sensors 114.

The opening 220 properly aligns the test sensor-extraction mechanism 208 with respect to the plurality of test sensors 114. As the slider 206 is moved in a forward direction, the test sensor-extraction mechanism 208 contacts and grips one of the plurality of test sensors 114 through the opening 120. As the slider 206 is moved to the second position (see FIG. 2c), the test sensor-extraction mechanism 208 continues to grip one of the plurality of test sensors 114 that is held down in place by the holding plate 126. After the test sensor-extraction mechanism 208 has gripped one of the plurality of test sensors 114, the slider 206 is moved back to the first position.

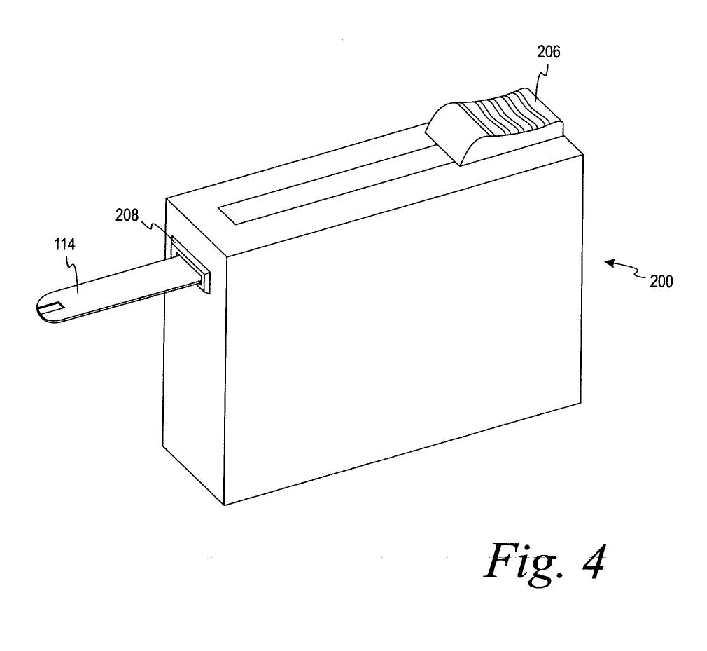

As the slider 206 moves back to the first position, the test sensor-extraction mechanism 208 continues to grip and pull one of the plurality of lest sensors 114 until the sensor has been separated from the stack of the plurality of test sensors 114 and has at least partially passed through the opening 120 (see FIGS. 3a,b). As the test sensor extraction mechanism 208 grips and pulls one of the plurality of test sensors 114, the inner cartridge 104 including the test sensors 114 begins to move towards the housing 204. After the slider 206 has returned to the first position, the test sensor-extraction mechanism 208 retains the sensor within its grasp and presents the sensor in a manner suitable for use by a user (see FIG. 4).

The movement of the inner cartridge 104 during the gripping and pulling of test sensor 114, in one embodiment, is stopped by the stop member 122 upon contacting an inner surface of the outer cartridge 102, which prevents or inhibits the inner cartridge from contacting the inner surface 102a of the outer cartridge (see FIG. 1a). In another embodiment, the movement of the inner cartridge 104 stops when the outer surface of the inner cartridge contacts the inner surface of the outer cartridge.

In one embodiment of the invention, the test sensor-extraction mechanism 208 comprises electrical contacts that link a sensor 114 to the meter electronics (not shown) contained within the housing 204. The sensor 114 may be linked to the meter electronics via sliding contacts or via flexible circuit cables (not shown).

The testing end of the sensor is adapted to be placed into contact with the fluid sample (e.g., a whole blood sample) to be tested. The whole blood sample may be generated by a lancing device such as a lancet. The whole blood sample may be obtained by a lancet that may be separate from the single-sensor meter system or may be integrated within the single-sensor meter. The lancing device may obtain blood by, e.g., pricking a person's finger.

According to one process, the whole blood sample may be prepared for testing by (a) advancing one of the test sensors in position to receive a whole blood sample; (b) generating a whole blood sample; and (c) bringing the test sensor and the whole blood sample into contact wherein the blood is generally drawn into the sensor by capillary action.

The sensors are typically provided with a capillary channel that extends from the front or testing end of the sensors to biosensing or reagent material disposed in the sensor. When the testing end of the sensor is placed into fluid (e.g., blood that is accumulated on a person's finger after the finger has been pricked), a portion of the fluid is drawn into the capillary channel by capillary action. In one method, the fluid then chemically reacts with the reagent material in the sensor so that an electrical signal indicative of the blood glucose level in the blood being tested is supplied and subsequently transmitted to an electrical assembly.

After the testing has been completed, the test sensor 114 may be removed by several methods from the housing 204. In one embodiment, the single-sensor meter may include an eject mechanism 232 that ejects the used test sensor from the single-sensor meter. In such an embodiment, the test sensor is released forcefully. In another embodiment, the test sensors may be ejected by releasing a grip of the test sensors; resulting in the test sensor being discarded by gravity from the single-sensor meter. In a further embodiment, the test sensor may also be removed manually from the single-sensor meter.

The test sensor-extraction mechanism 208 extends through the opening 120 in the container assembly 100 when being moved to the second position. In this extended position, the test sensor-extraction mechanism 208 contacts and grips one of the test sensors 114.

When the slider 206 is moved in a backward direction (direction of arrow C shown in FIG. 2c) from its second position to the first position of FIG. 2a, the test sensor-extraction mechanism 208 is simultaneously moved from its second position to the first position. This results in the test sensor-extraction mechanism 208 passing through the opening 120. While the slider 206 and the test sensor-extraction mechanism 208 are in the first position, the container assembly 100 is substantially moisture-proof and air-tight when the end cap is in the closed position.

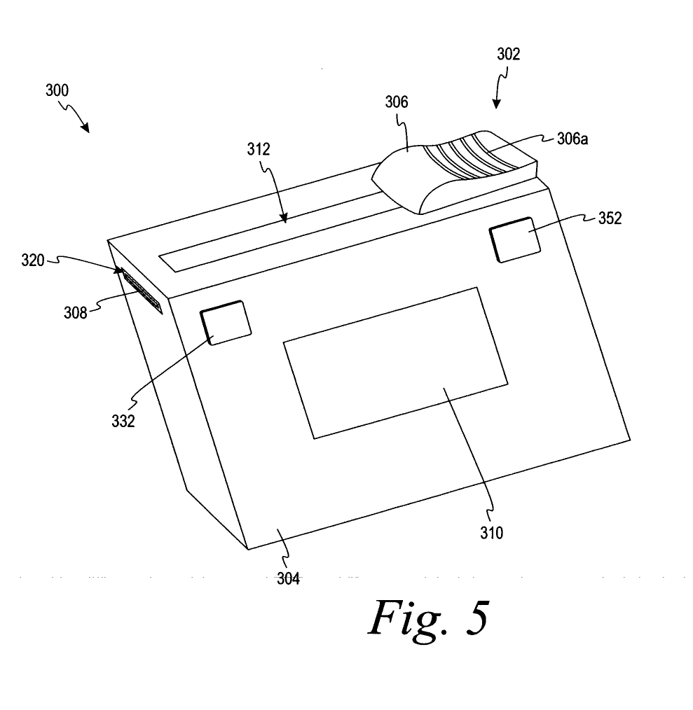

It is also contemplated that the single-sensor meter may activate the slider mechanism automatically such as in response to pressing a button. For example, referring to FIG. 5, a single-sensor meter system 300 includes a mechanism such as button 352 to activate the slider mechanism. The single-sensor meter 300 includes sliding assembly 302, housing 304, slider 306, test sensor-extraction mechanism 308, LCD screen 310, internal channel 312, opening 320 and eject mechanism 332. Other than the activation mechanism for the slider mechanism (button 352), the single-sensor meter 300 functions similar to the single-sensor meter 200 as described above.

The housing 204 and the slider 206 are typically made of a polymeric materials. Non-limiting examples of polymeric materials include polycarbonate, ABS, nylon, polypropylene, or combinations thereof. Additives may be added to the polymeric material that forms the slider. It is contemplated that the slider may be made of other materials such as metallic materials.

The test sensor-extraction mechanism 208 may be made of metal or polymeric material. Some non-limited metallic materials include stainless steel and bronze with appropriate plating. Non-limiting examples of polymeric materials include polycarbonate, ABS, nylon, polypropylene, or combinations thereof. Additives may be added to the polymeric material that forms the test sensor-extraction mechanism.

The single-sensor meter 200 typically includes a microprocessor or the like for processing and/or storing data generated during the blood glucose test procedure. This data may be displayed on the liquid crystal display 210 located on the surface of the housing 204 (see FIG. 2a). The liquid crystal display displays information from the testing procedure on the single-sensor meter 200.

Some of the information that may be displayed when the single-sensor meter is in use include the following: a battery indication, a numerical display, apply blood indication, a temperature indication, meal markers, or various combinations thereof. The numerical display typically shows testing results including, but not limited to, specific analyte concentration readings, average analyte concentrations, and high and low analyte concentrations.

The single-sensor meter 200 may also contain an opening for a battery-tray assembly. The battery-tray assembly includes a battery-tray in which a battery is disposed. The battery-tray assembly is inserted into the opening in a side of the single-sensor meter 200. When so inserted, the battery provides power for the electronics within the single-sensor meter 200, including the circuitry on the circuit board assembly (not shown) and the liquid crystal display 210.

While the invention has been described with reference to details of the illustrated embodiment, these details are not intended to limit the scope of the invention as defined in the appended claims. For example, the single-sensor meter 200 may be used for testing fluids other than blood glucose. In fact, the single-sensor meter 200 may be used in connection with the analysis of any type of chemistry fluid that can be analyzed by using a reagent material.

### Alternative Embodiment A

A single-sensor meter system for dispensing sensors for testing of an analyte concentration in a fluid, the meter system comprising:

a container assembly including an inner cartridge and an outer cartridge, the inner cartridge including a plurality of test-sensors and a mechanical mechanism, the container assembly forming exactly one opening for dispensing the test sensors one at a time, the opening being covered by an end cap so as to assist in preventing or inhibiting moisture from entering the interior of the container assembly, the mechanical mechanism being adapted to advance the plurality of test sensors; and

a single-sensor meter being adapted to align with and operatively connects to the container assembly, the single-sensor meter including a test-sensor extraction mechanism adapted to grip a test sensor and pull the test sensor through the opening to a dispensed position.

### Alternative Embodiment B

The single-sensor meter system of embodiment A wherein the plurality of test sensors is from about 10 to about 100 test sensors.

### Alternative Embodiment C

The single-sensor meter system of embodiment B wherein the plurality of test sensors is from about 25 to about 50 test sensors.

### Alternative Embodiment D

The single-sensor meter system of embodiment A wherein the mechanical mechanism is at least one spring.

### Alternative Embodiment E

The single-sensor meter system of embodiment D wherein the mechanical mechanism is a plurality of springs.

### Alternative Embodiment F

The single-sensor meter system of embodiment A wherein the plurality of test sensors is electrochemical-based test sensors and the single-sensor meter is adapted to read the electrochemical-based test sensors.

### Alternative Embodiment G

The single-sensor meter system of embodiment A wherein the plurality of test sensors is optical-based test sensors and the single-sensor meter is adapted to read the optical-based test sensors.

### Alternative Embodiment H

The single-sensor meter system of embodiment A wherein the end cap is removable.

### Alternative Embodiment I

The single-sensor meter system of embodiment A wherein the end cap is adapted to pivot between an open position and a closed position.

### Alternative Embodiment J

The single-sensor meter system of embodiment A wherein the end cap is not adapted to be removed from the container assembly.

### Alternative Embodiment K

The single-sensor meter system of embodiment A wherein the single-sensor meter further includes a sensor-eject mechanism.

### Alternative Embodiment L

The single-sensor meter system of embodiment A wherein one of the test sensors is a calibration test sensor.

### Alternative Embodiment M

The single-sensor meter system of embodiment A wherein the test sensor-extraction member is reciprocally slidable between a first position and a second position.

### Alternative Process N

A method of operating a single-sensor meter system to determine an analyte concentration of a fluid, the method comprising the acts of:

providing a container assembly including an inner cartridge and an outer cartridge, the inner cartridge including a plurality of test sensors and a mechanical mechanism, the container assembly forming exactly one opening for dispensing the test sensors one at a time, the opening being covered by an end cap so as to assist in preventing or inhibiting moisture from entering the interior of the container assembly, the mechanical mechanism being adapted to advance the plurality of test sensors;

providing a single-sensor meter including a test-sensor extraction mechanism;

moving the end cap from the closed position to the open position;

aligning the container assembly and the single-sensor meter;

retrieving one of the plurality of test sensors from the container assembly such that one of the test sensors is at least partially located within the single-sensor meter; and

determining the concentration of the analyte.

### Alternative Process O

The method of process N wherein the retrieving of one of the plurality of test sensors includes activating a meter button such that the test sensor extraction mechanism extends into the opening of the container assembly and retrieves one of the plurality of test sensors.

### Alternative Process P

The method of process N wherein the analyte is glucose.

### Alternative Process Q

The method of process N wherein the fluid is blood.

### Alternative Process R

The method of process N wherein the single-sensor meter further includes a sensor-eject mechanism, and further including ejecting the test sensor from the single-sensor meter via the sensor-eject mechanism.

### Alternative Process S

The method of process N wherein the test sensor-extraction mechanism is manually moved from a first position to a second position by a user.

### Alternative Process T

The method of process N wherein the test sensor-extraction mechanism is automatically moved from a first position to a second position by a user.

### Alternative Process U

The method of process N further including moving the end cap to a closed position.

### Alternative Process V

The method of process N wherein the mechanical mechanism is at least one spring.

### Alternative Process W

The method of process V wherein the mechanical mechanism is a plurality of springs.

### Alternative Process X

The method of process N wherein the plurality of test sensors is electrochemical-based test sensors and the single-sensor meter is adapted to read the electrochemical-based test sensors.

### Alternative Process Y

The method of process N wherein the plurality of test sensors is optical-based test sensors and the single-sensor meter is adapted to read the optical-based test sensors.

### Alternative Process Z

The method of process N wherein the end cap is removable.

### Alternative Process AA

The method of process N wherein the end cap is adapted to pivot between an open position and a closed position.

### Alternative Process BB

The method of process N wherein the end cap is not adapted to be removed from the container assembly.

### Alternative Process CC

The method of process N wherein one of the test sensors is a calibration test sensor.

### Alternative Process DD

The method of process N wherein the test sensor-extraction member is reciprocally slidable between a first position and a second position.

While the invention is susceptible to various modifications and alternative forms, specific embodiments are shown by way of example in the drawings and described in detail. It should be understood, however, that it is not intended to limit the invention to the particular forms disclosed, but on the contrary, the intention is to cover all modifications, equivalents, and alternatives falling within the spirit and scope of the invention as defined by the appended claims.

1. A single-sensor meter system for dispensing sensors for testing of an analyte concentration in a fluid, the meter system comprising:
a container assembly including an inner cartridge and an outer cartridge, the inner cartridge including a plurality of test sensors and a mechanical mechanism, the container assembly forming exactly one opening for dispensing the test sensors one at a time, the opening being covered by an end cap so as to assist in preventing or inhibiting moisture from entering the interior of the container assembly, the mechanical mechanism being adapted to advance the plurality of test sensors; and
a single-sensor meter being adapted to align with and operatively connects to the container assembly, the single-sensor meter including a test-sensor extraction mechanism adapted to grip a test sensor and pull the test sensor through the opening to a dispensed position.

2. The single-sensor meter system of claim 1, wherein the plurality of test sensors is from about 10 to about 100 test sensors.

3. (canceled)

4. The single-sensor meter system of claim 1, wherein the mechanical mechanism is at least one spring.

5. (canceled)

6. The single-sensor meter system of claim 1, wherein the plurality of test sensors is electrochemical-based test sensors and the single-sensor meter is adapted to read the electrochemical-based test sensors.

7. The single-sensor meter system of claim 1, wherein the plurality of test sensors is optical-based test sensors and the single-sensor meter is adapted to read the optical-based test sensors.

8. The single-sensor meter system of claim 1, wherein the end cap is removable.

9. The single-sensor meter system of claim 1, wherein the end cap is adapted to pivot between an open position and a closed position.

10. The single-sensor meter system of claim 1, wherein the end cap is not adapted to be removed from the container assembly.

11-12. (canceled)

13. The single-sensor meter system of claim 1, wherein the test sensor-extraction member is reciprocally slidable between a first position and a second position.

14. A method of operating a single-sensor meter system to determine an analyte concentration of a fluid, the method comprising the acts of:
providing a container assembly including an inner cartridge and an outer cartridge, the inner cartridge including a plurality of test sensors and a mechanical mechanism, the container assembly forming exactly one opening for dispensing the test sensors one at a time, the opening being covered by an end cap so as to assist in preventing or inhibiting moisture from entering the interior of the container assembly, the mechanical mechanism being adapted to advance the plurality of test sensors;
providing a single-sensor meter including a test-sensor extraction mechanism;
moving the end cap from the closed position to the open position;
aligning the container assembly and the single-sensor meter;
retrieving one of the plurality of test sensors from the container assembly such that one of the test sensors is at least partially located within the single-sensor meter; and
determining the concentration of the analyte.

15. The method of claim 14, wherein the retrieving of one of the plurality of test sensors includes activating a meter button such that the test sensor extraction mechanism extends into the opening of the container assembly and retrieves one of the plurality of test sensors.

16. The method of claim 14, wherein the analyte is glucose.

17. (canceled)

18. The method of claim 14, wherein the single-sensor meter further includes a sensor-eject mechanism, and further including ejecting the test sensor from the single-sensor meter via the sensor-eject mechanism.

19. The method of claim 14, wherein the test sensor-extraction mechanism is manually moved from a first position to a second position by a user.

20. The method of claim 14, wherein the test sensor-extraction mechanism is automatically moved from a first position to a second position by a user.

21-23. (canceled)

24. The method of claim 14, wherein the plurality of test sensors is electrochemical-based test sensors and the single-sensor meter is adapted to read the electrochemical-based test sensors.

25. The method of claim 14, wherein the plurality of test sensors is optical-based test sensors and the single-sensor meter is adapted to read the optical-based test sensors.

26. The method of claim 14, wherein the end cap is removable.

27. The method of claim 14, wherein the end cap is adapted to pivot between an open position and a closed position.

28-29. (canceled)

30. The method of claim 14, wherein the test sensor-extraction member is reciprocally slidable between a first position and a second position.
